(19) United States
(12) Patent Application Publication
(10) Pub. No.: US 2005/0107859 A1
(43) Pub. Date: May 19, 2005

(54) System and method of establishing an electrical connection between an implanted lead and an electrical contact

(76) Inventors: Terry Daglow, Allen, TX (US); Robert Jones, McKinney, TX (US)

Correspondence Address: Koestner Bertani, LLP, P.O. Box 26780, Austin, TX, 78755, US

(21) Appl. No.: 10/713,286

(22) Filed: Nov. 14, 2003

(51) Int. Cl. A61N 1/18; A61N 1/375

(52) U.S. Cl. 607/116; 607/36

(57) ABSTRACT

An electrical contact to electrically couple epidural stimulation leads to a tissue stimulation device. This electrical contact includes a conductive disc having an inner aperture that is electrically and operably coupled to the tissue stimulation device. A number of conducted projections extend inward from the inner aperture of the disc and flex elastically to receive an epidural stimulation lead. These conductive projections electrically couple with a number of terminals within the epidural stimulation lead. Tension exerted by flexing the conducted projections maintains the epidural stimulation lead in a relatively fixed position relative to the electrical contact. A number of projections at the outer perimeter of the conductive disc electrically and operably couple the conductive disc to a conductive housing wherein the conductive housing is electrically and operably coupled to the tissue stimulation device.

### TECHNICAL FIELD OF THE INVENTION

The present invention relates generally to electrical contacts, and more particularly, a system and method for electrically coupling an implanted lead with an electrical contact.

### BACKGROUND OF THE INVENTION

Electrical stimulation of the spinal cord or peripheral nerves can result in pain reduction and/or elimination. Medical leads having electrodes are often implanted near the spinal column to provide pain relief for chronic intractable pain. The electrodes stimulate tissue within the spinal column to reduce pain sensations at other parts of the body. The stimulation signals applied can be optimized for pain reduction or elimination depending on the location of the pain.

The area of excitation and the intensity of excitation vary according to the stimulation signals. To vary the area of excitation, an array of electrodes implanted near nerve tissue can be configured for a positive, negative, or neutral polarity such that the desired area within the spinal column is electrically stimulated. In addition, the stimulation signal applied on those implanted electrodes can be varied for a corresponding variation in area of excitation within the spinal column and in the intensity of excitation at the pain site.

Leads are typically connected to a tissue stimulator through a header that contains various connector assembles. These prior art connectors often fail to consistently provide adequate electrical coupling with the lead or are difficult to connect to the lead. For example, FIG. 1 shows a prior art electrical connection, which would be located in a header of a tissue stimulator and is used to electrically couple the contacts on a lead to terminals located within the header of the tissue stimulator. Here, contact 10, which may be contained in the header which is in the inert housing of the tissue stimulator, not shown, has in a cylindrical band 12 having an inner groove bounded by lower lip 14 and upper lip 16. The groove bounded by the upper and lower lip in this instance contains a coiled spring, which contacts (electrically couples) the terminals of the implantable lead when the lead passes through contact 10. Some problems associated with this solution are the difficulty in machining the upper and lower lip to retain the contact spring 18, as well as placing the spring within band 12. Associated with these difficulties is a high cost of manufacturing.

Another embodiment uses individual setscrews to electrically couple contacts and leads. This process is difficult and time consuming. This is especially true in a surgical environment when rework would require additional surgery. This surgery places undue physical, mental, and financial strains on the patient.

### SUMMARY OF THE INVENTION

The present invention provides a system of establishing an electrical connection between an implantable lead and an electrical contact that substantially eliminates or reduces disadvantages and problems associated with previously developed solutions. More specifically, this disclosure teaches an electrical contact that electrically couples an implantable lead to a tissue stimulation device.

This contact includes a conductive disc having an inner aperture wherein the conductive disc is electrically and operatively coupled to the tissue stimulation device. A number of conductive projections (or fingers) extend inwards from the perimeter of the inner aperture of the conductive disc. These fingers receive the implantable lead allowing an electrical connection to be made between terminals on the implantable lead and the conductive disc via the conductive projections. The conductive projections flex thereby exerting tension on the implantable lead. This tension serves to maintain the implantable lead in a relatively fixed position relative to the conductive disc.

Another embodiment may include a number of projections located at the outer perimeter of the conductive disc to electrically and operably couple the conductive disc to a conductive housing which is again, electrically and operably coupled to a tissue stimulation device.

The implantable lead may correspond to an epidural stimulation device that is used to provide a stimulation pattern from an applied electric field. Such an epidural stimulation lead typically includes an inert body having a surface on which a number of electrodes are positioned, a number of terminals that electrically and operably connect the epidural stimulation lead to an external source used to provide the electrical stimulation signal. A number of conductors connect each terminal to at least one electrode allowing the electrical signal to have a circuit path from the tissue stimulation device to the end of the epidural stimulation lead in order to provide an applied electric field.

Another embodiment contained in this disclosure teaches a neuromodulation therapy system. In this instance, an implantable stimulation lead delivers electrical energy to tissues located proximately to the stimulation lead. An implantable receiver is operably and electrically coupled to the stimulation lead and delivers electrical energy in the form of a stimulation signal to the stimulation lead in response to a control signal. An electrical contact, operably and electrically couples the implantable stimulation lead to the implantable receiver.

As previously discussed, this contact includes a conductive disc having an inner aperture wherein the conductive disc is electrically and operatively coupled to the tissue stimulation device. A number of conductive projections or fingers extend inwards from the perimeter of the inner aperture of the conductive disc and receive the implantable lead allowing an electrical connection to be made between terminals on the implantable lead and the conductive disc via the conductive projections. The fingers flex to retain the implantable lead. This tension serves to maintain the implantable lead in a relatively fixed position relative to the conductive disc.

Another embodiment teaches a method of operably coupling an implantable lead to a tissue stimulation device with a contact. This contact operably couples the conductive disc having an inner aperture to a tissue stimulation device. The conductive disc receives an implantable lead with a plurality of conductive projections that extend inwards from the inner aperture of the disc. These projections flex and electrical couple to a number of terminals within the implantable lead. Tension exerted by flexing the conducted projections maintains the implantable lead in a relatively fixed position relative to the first set of electrical terminals.

### BRIEF DESCRIPTION OF THE DRAWINGS

For a more complete understanding of the present invention and the advantages thereof, reference is now made to the following description taken in conjunction with the accompanying drawings in which like reference numerals indicate like features and wherein:

FIG. 1 illustrates a prior art contact used to operably couple an implantable stimulation lead with an implantable receiver;

FIG. 2 provides an isometric view of a disc shaped contact as taught in this disclosure;

FIG. 3 provides an isometric view of a conductive housing which mechanically and electrically couples the electrical contact of FIG. 2 with an implantable receiver or other like device;

FIG. 4 provides a pictorial view of the electrical contact of FIG. 2 mechanically and electrically coupled to the conductive housing of FIG. 3;

FIGS. 5A, 5B, and 5C depict the contact of FIGS. 2-4 located within a header; FIG. 6 depicts a number of contacts electrically coupled to terminals within the stimulation lead;

FIG. 7 provides a diagram functionally illustrating the implantable lead electrically coupled to the contacts taught in this disclosure; and

FIG. 8 provides a systems overview of a tissue stimulation system that receives an external signal from a processor to generate a stimulation signal, which is passed to the implantable lead through an electrical connection between conductors from the implantable receiver to the implantable lead.

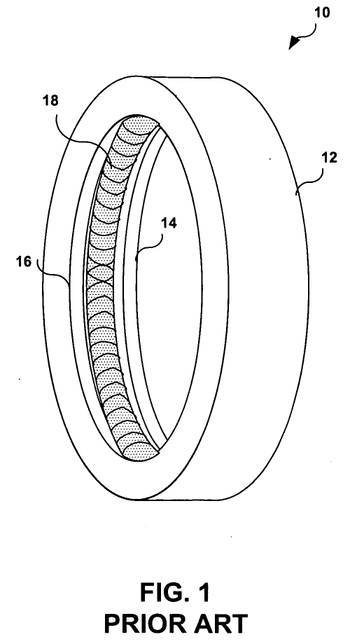

### DETAILED DESCRIPTION OF THE INVENTION

Preferred embodiments of the present invention are illustrated in the figures, like numerals being used to refer to like and corresponding parts of the various drawings

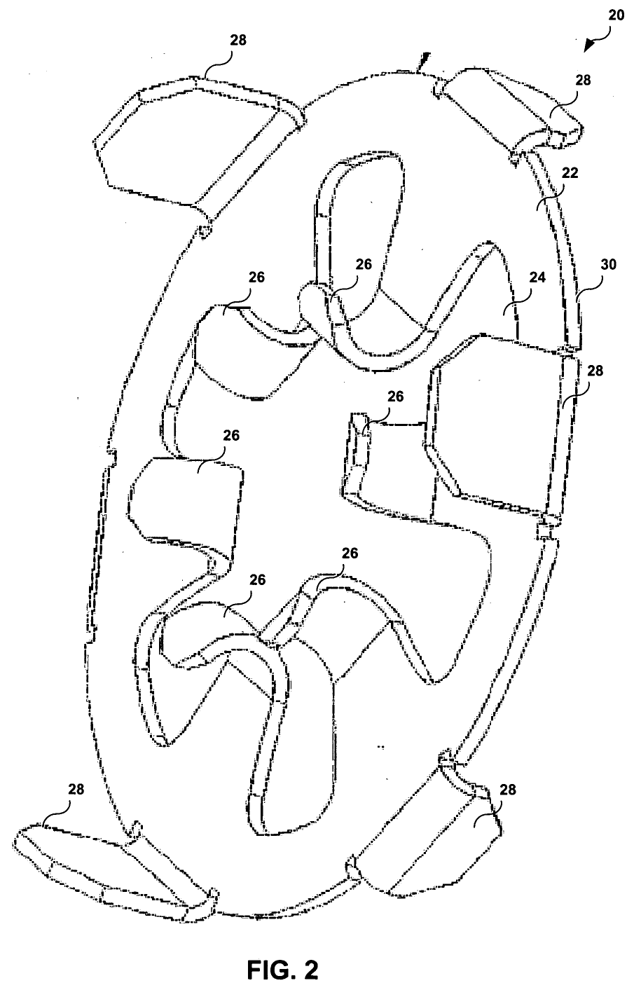

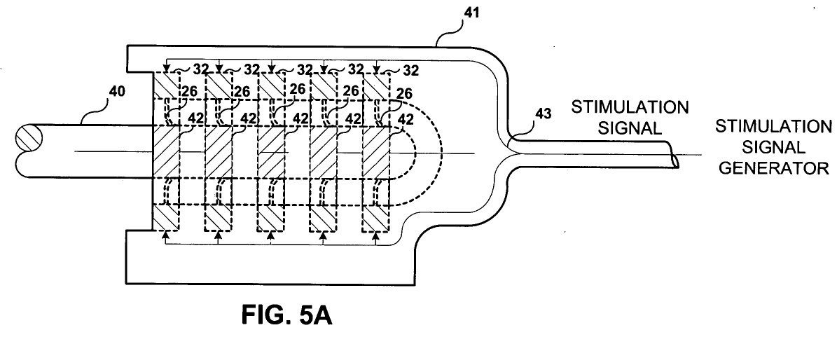

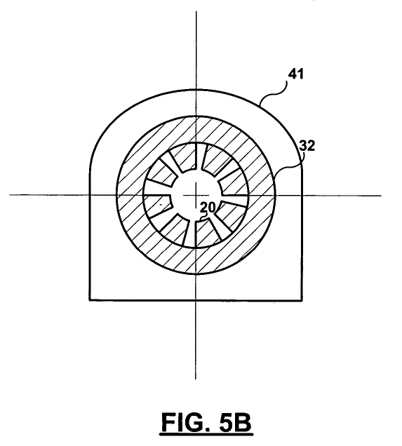

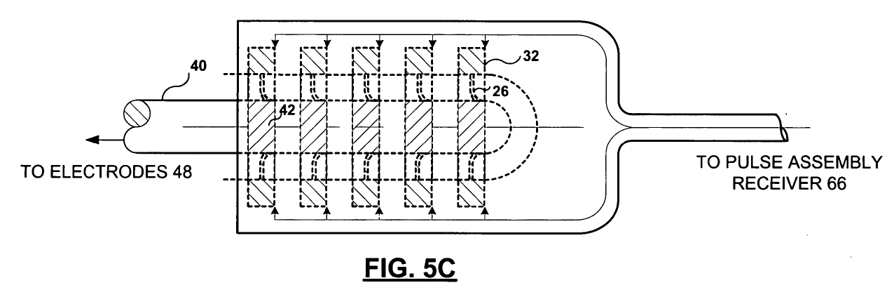

FIG. 2 provides a pictorial representation of electrical contact 20 that greatly reduces the cost of manufacturing and simplifies the surgical implantation. The contact forms part of the electrical circuit that operably couples an implantable lead and a controller or processing module. The implantable lead will be discussed in further detail as depicted in FIG. 5. The contact operably couples the implantable lead to a tissue stimulation device.

The electrical contact is formed from a conductive disc 22 having an inner aperture 24 and conductive projections 26 (or fingers) that extend away from the inner aperture. The number of conductive projections corresponds to the number of contacts on the lead, which may correspond to the number of electrodes located on the lead.

The conductive disc 22 is located within the header of a tissue stimulator. This header is better depicted in FIGS. 5A-5C. Conductive disc is connected to the tissue stimulated using feed-throughs thereby ensuring an electrical connection between the tissue stimulator and the implantable lead.

In one embodiment, conductive projections 26 are flexible and are shaped to receive the contacts from an implantable lead (not shown) and enforce an electrical coupling of the contacts to the conductive disc 22. The tension resulting from flexing conductive projections 26 as the implantable led deflects the conductive projections outwards maintains the implantable lead in a relatively fixed position relative to the conductive disc. In this case, conductive projections 26 may be made of a memory shaped alloy, such as Nitinol. Such a memory shaped alloy may be heated or cooled to maintain sufficient mechanical contact force. The memory shaped alloy can be coated with a highly conductive material such as platinum-iridium to maximize electrical conductivity of the connection.

In another embodiment, the conductive projections 26 may flex elastically allowing the contacts to be disconnected and reconnected with the implantable lead. In this case, a setscrew might be used to hold the lead in place within the header.

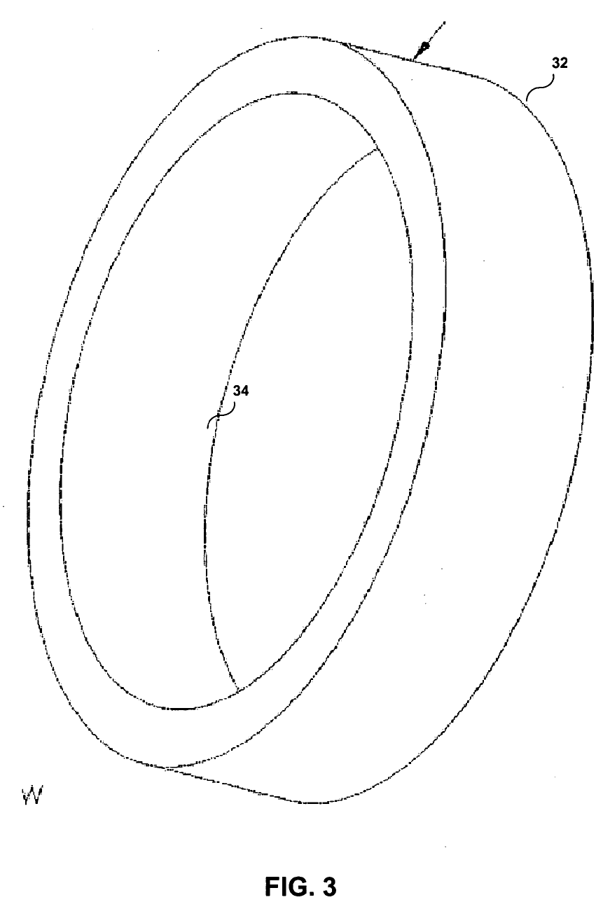

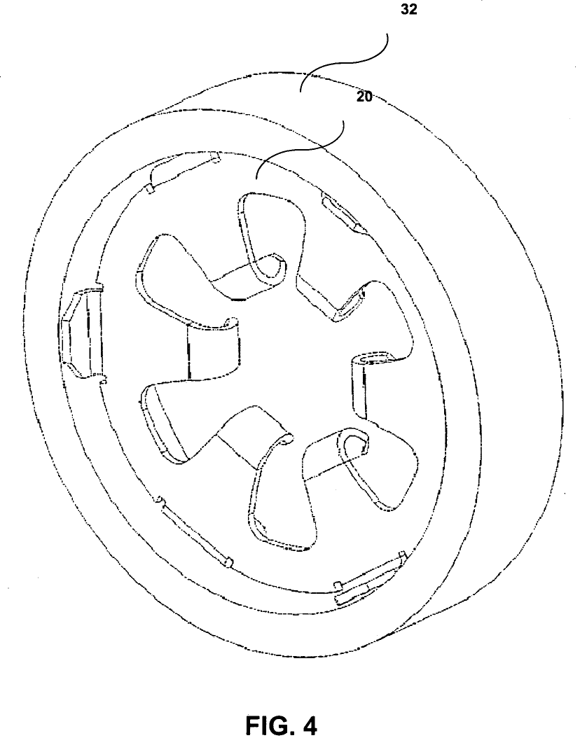

In one embodiment, outer projections 28, located at outer perimeter 30 of conductive disc 22 operably couple the conductive disc to a conductive housing 32 depicted in FIG. 3. Inner surface 34 of conductive housing 32 receive projections 28. FIG. 4 depicts conductive disc 22 pressed into or mechanically and electrically affixed to conductive housing 32. However, those skilled in the art will recognize that conductive disc 22 can be manufactured in such a way that it and conductive housing 32 are stamped or formed from one piece, thereby negating the need for outer projections 28. FIGS. 5A, 5B, and 5C illustrate header 41, which receives an implantable lead 40. FIG. 5A provides a cross-sectional view of header 41. FIG. 5B provides a front view of header 41. FIG. 5C provides a top-down view again of header 41. As can be clearly seen here, lead 40 with contacts 42 is received within header 41. Contacts 20 are pressed into place within the conductive housing's 32, and have projections 26 which electrically couple contacts 42 to the stimulation signal provided by the conductive leads 43 that couple individual conductive housings 32 to a stimulation signal generator. FIG. 5A and FIG. 5C clearly show that projection's 26 may deflect as the implantable lead is received within header 41. Their deflection creates tension that maintains the implantable lead 40 in a pre-determined position relative to header 41.

Individual connectors 20 receive an implantable lead 40 as shown in FIGS. 5A-5C and FIG. 6. Implantable lead 40 will have contacts that make contact with the connectors to ensure an electrical connection. Here, connectors 20 are contained within an inert implantable housing 41 that receives implantable lead 40. As shown here, conductive projections 26 couple to terminals 42 within implantable lead 40.

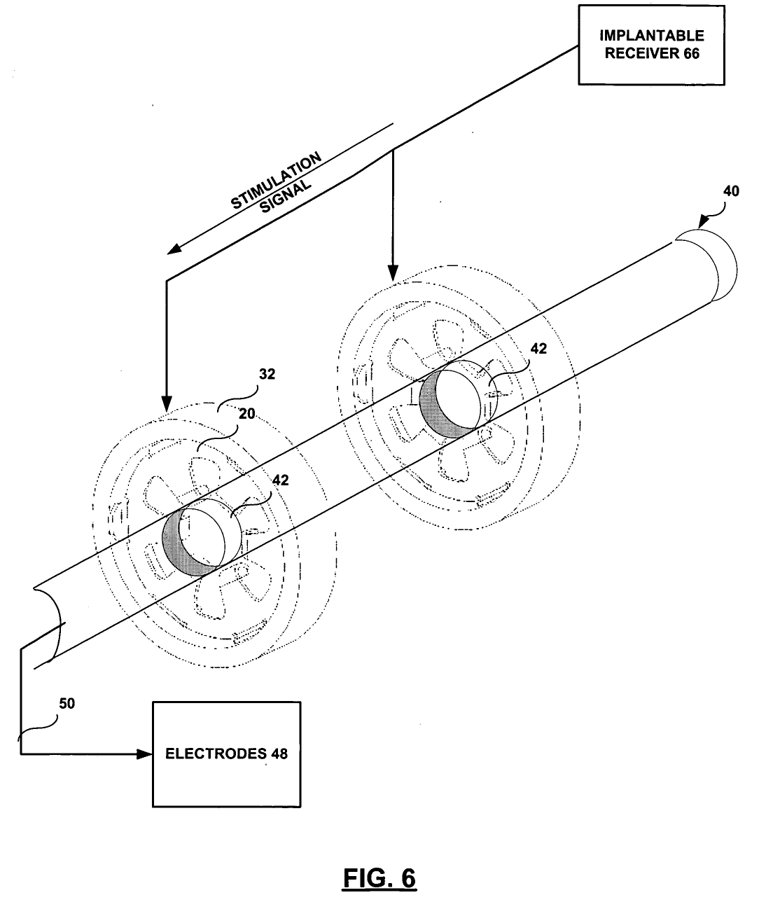

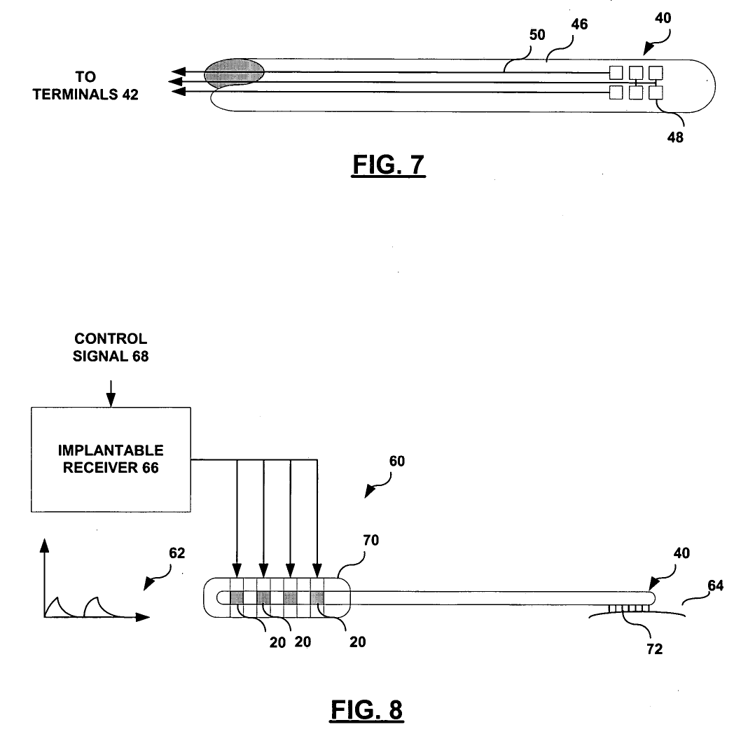

FIG. 7 shows in further detail implantable lead 40, which is made up of an inert body 46 and has a number of electrodes 48 positioned relative to the surface 46. A number of conductors 50 couple electrodes 48 to terminals 42 as previously discussed. Although a generic lead is shown, other leads known to those skilled in the art may be used. These include, but are not limited to, any and all manner of lamitrodes and percutanous leads.

FIG. 8 shows a neuromodulation system or tissue stimulator system 60 used to apply electrical stimulation in the form of electrical signal 62 to tissue 64. Implantable lead 40 is located proximate to tissue 64 and electrically and operably coupled to a controller or processor. Implantable receiver 66 may be physically or logically located within a controller or processor 70.

Implantable receiver 66 receives a control signal 68 from the processor. Upon receiving control signal 68, receiver (or pulse generator) 66 produces electrical stimulation signal 62 for implantable stimulation lead 40 that travels through inert housing 70 or header, which actually receives the stimulation lead. This electrical stimulation signal may take the form of a series of generator electrical pulses.

Controller or processor 70 may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. Memory operably coupled to the processor may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the controller or processor 70 implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The memory stores, and the controller or processor 70 executes operational instructions to the functions of the neuromodulation system.

In summary, the above discussion teaches an electrical contact to electrically couple an epidural stimulation lead to a tissue stimulation device. This electrical contact includes a conductive disc having an inner aperture that is electrically and operably coupled to the tissue stimulation device. A number of conducted projections extend inward from the inner aperture of the disc and flex elastically to receive an epidural stimulation lead. These conductive projections electrically couple with a number of terminals within the epidural stimulation lead. Tension exerted by flexing the conducted projections maintains the epidural stimulation lead in a relatively fixed position relative to the electrical contact. A number of projections at the outer perimeter of the conductive disc electrically and operably couple the conductive disc to a conductive housing wherein the conductive housing is electrically and operably coupled to the tissue stimulation device.

As one of average skill in the art will appreciate, the term “substantially” or “approximately”, as may be used herein, provides an industry-accepted tolerance to its corresponding term. Such an industry-accepted tolerance ranges from less than one percent to twenty percent and corresponds to, but is not limited to, component values, integrated circuit process variations, temperature variations, rise and fall times, and/or thermal noise. As one of average skill in the art will further appreciate, the term “operably coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. As one of average skill in the art will also appreciate, inferred coupling (i.e., where one element is coupled to another element by inference) includes direct and indirect coupling between two elements in the same manner as “operably coupled”. As one of average skill in the art will further appreciate, the term “compares favorably”, as may be used herein, indicates that a comparison between two or more elements, items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

Although the present invention is described in detail, it should be understood that various changes, substitutions and alterations can be made hereto without departing from the spirit and scope of the invention as described by the appended claims.

1. An electrical contact to electrically couple an implantable lead to a tissue stimulation device, comprising:
a conductive disk having an inner aperture, wherein the conductive disk is operably coupled to the tissue stimulation device; and
a plurality of conductive projections extending away from the inner aperture of the disk, wherein the conductive projections flex to receive the implantable lead and electrically couple to a plurality of terminals within the implantable lead.

2. The electrical contact in claim 1 wherein a tension exerted by flexing the conductive projections maintains the implantable lead relative to the first set of electrical terminals.

3. The electrical contact of claim 1, wherein the conductive projections flex elastically.

4. The electrical contact of claim 1, further comprising a plurality of projections at the outer perimeter of the conductive disk which operable couple the conductive disk to a housing wherein the housing is operable coupled to the tissue stimulation device.

5. The electrical contact of claim 1, wherein conductive projections are comprised of a shape memory alloy.

6. The electrical contact of claim 5, wherein the stimulation lead provides a stimulation pattern from an applied electric field and comprises:
a body having a first surface;
a plurality of electrodes positioned relative to the first surface of the body; and
a plurality of conductors, wherein a conductor electrically couples one terminal of the plurality of terminals with at least one electrode.

7. The electrical contact of claim 1, wherein the tissue stimulation device comprises an implantable receiver to deliver electrical stimulation signals to the implantable lead in response to a control signal.

8. A neuromodulation therapy system comprising:
at least one implantable stimulation lead to deliver of electrical energy to proximately positioned tissue;
an implantable pulse generator coupled to the at least one stimulation lead, wherein the implantable pulse generator is operable to deliver electrical energy to the at least one stimulation lead in response to at least one control signal, and wherein an electrical contact operably couples the at least one implantable stimulation lead to the implantable receiver, the electrical contact comprising:
a conductive disk having an inner aperture, wherein the conductive disk is operably coupled to the implantable receiver; and
a plurality of conductive projections extending away from the inner aperture of the disk, wherein the conductive projections flex to receive the at least one implantable stimulation lead and electrically couple to a plurality of terminals within the at least one implantable stimulation lead.

9. The system of claim 8, wherein tension exerted by flexing the conductive projections maintains the at least one implantable stimulation lead relative to the first set of electrical terminals.

10. The neuromodulation therapy system of claim 8, wherein the conductive projections of the electrical contact flex elastically.

11. The neuromodulation therapy system of claim 8, wherein the electrical contact, further comprises a plurality of projections at the outer perimeter of the conductive disk which operable couple the conductive disk to a housing wherein the housing is operable coupled to the implantable receiver.

12. The neuromodulation therapy system of claim 8, wherein the projections are comprised of a shape memory alloy.

13. The neuromodulation therapy system of claim 11, wherein the stimulation lead provides a stimulation pattern from an applied electric field and comprises:
a body having a first surface;
a plurality of electrodes positioned relative to the first surface of the body; and
a plurality of conductors, wherein a conductor electrically couples one terminal of the plurality of terminals with at least one electrode.

14. A method to operably couple an implantable lead to a tissue stimulation device that comprises:
operably coupling a conductive disk having an inner aperture to the tissue stimulation device; and
receiving the implantable lead with a plurality of conductive projections extending away from the inner aperture of the disk, wherein the conductive projections flex and electrically couple to a plurality of terminals within the implantable lead.

15. The method of claim 14, wherein tension exerted by flexing the conductive projections maintains the implantable lead relative to the first set of electrical terminals.

16. The method of claim 14, wherein the conductive projections flex elastically.

17. The method of claim 14, wherein operably coupling the conductive disk to the tissue stimulation device further comprises operable coupling a plurality of projections at the outer perimeter of the conductive disk to a housing wherein the housing is operable coupled to the tissue stimulation device.

18. The method of claim 14, wherein the projections are comprised of a shape memory alloy.

19. The method of claim 14, wherein the stimulation lead provides a stimulation pattern from an applied electric field and comprises:
a body having a first surface;
a plurality of electrodes positioned relative to the first surface of the body; and
a plurality of conductors, wherein a conductor electrically couples one terminal of the plurality of terminals with at least one electrode.

20. The method of claim 14, wherein the tissue stimulation device comprises an implantable receiver to deliver electrical stimulation signals to the implantable lead in response to a control signal.

21. An electrical contact to electrically couple an epidural stimulation lead to a tissue stimulation device, comprising:
a conductive disk having an inner aperture, wherein the conductive disk is operably coupled to the tissue stimulation device;
a plurality of conductive projections extending inwards from the inner aperture of the disk, wherein the conductive projections flex elastically to receive the epidural stimulation lead and electrically couple to a plurality of terminals within the epidural stimulation lead, and wherein tension exerted by flexing the conductive projections maintains the epidural stimulation lead relative to the first set of electrical terminals; and
a plurality of projections at the outer perimeter of the conductive disk which operable couple the conductive disk to a housing wherein the housing is operably coupled to the tissue stimulation device.

22. The electrical contact of claim 21, wherein the epidural stimulation lead provides a stimulation pattern from an applied electric field and comprises:
a body having a first surface;
a plurality of electrodes positioned relative to the first surface of the body; and
a plurality of conductors, wherein a conductor electrically couples one terminal of the plurality of terminals with at least one electrode.

23. The electrical contact of claim 22, wherein the tissue stimulation device comprises an implantable receiver to deliver electrical stimulation signals to the implantable lead in response to a control signal.
